(19) United States
(12) Patent Application Publication
(10) Pub. No.: US 2014/0154312 A1
(43) Pub. Date: Jun. 5, 2014

(54) ORAL TARGETTED DRUG DELIVERY SYSTEM

(71) Applicants: Monica Gulati, Phagwara (IN); Sima Singh, Phagwara (IN); Sanjiv Duggal, Phagwara (IN); Rahul Satyakam, Mohali (IN); Mamta Sharma, Mohali (IN)

(72) Inventors: Monica Gulati, Phagwara (IN); Sima Singh, Phagwara (IN); Sanjiv Duggal, Phagwara (IN); Rahul Satyakam, Mohali (IN); Mamta Sharma, Mohali (IN)

(21) Appl. No.: 13/909,159

(22) Filed: Jun. 4, 2013

Related U.S. Application Data
(63) Continuation of application No. 13/824,509, now abandoned.

(30) Foreign Application Priority Data
Sep. 17, 2010 (IN) 2220/DEL/2010

(51) Int. Cl. A61K 9/00; A61K 35/74; A61K 45/06; A61K 31/513

(52) U.S. Cl. CPC A61K 9/0056; A61K 31/513; A61K 35/745; A61K 45/06
USPC 424/452; 424/93.4

(57) ABSTRACT

The present invention discloses an “Improved Oral Targetted Drug Delivery System (O-TDDS)” particularly suited for delivery of drugs having activity against the diseases located in the colon e.g. colon cancer, ulcerative colitis, protozoal infections etc. The system comprises two elements or parts viz. microspheres (drug+natural polymers such as guar gum or xanthan gum) and probiotics. Both the elements are packed together in a single, pharmaceutically acceptable oral dosage form such as a capsule. The system offers distinct advantages of drug delivery without undesirable side-effects of diarrhea, nausea or vomiting commonly encountered in case of anti-cancer drugs such as 5-Fluorouracil.

This Application is a continuation of International Application PCT/IN2011/00642 (U.S. National Phase 13/824,509) filed 19 Sep. 2011 for IMPROVED ORAL TARGETTED DRUG DELIVERY SYSTEM, the contents of which are herein incorporated by reference. Application PCT/IN2011/00642 claims foreign priority to Application 2220/DEL/2010 filed 17 Sep. 2010, the contents of which is hereby incorporated by reference.

### FIELD OF INVENTION

The field of invention pertains to pharmaceutical formulations. More specifically, it pertains to an “Improved Oral Targetted Drug Delivery System (O-TDDS)”. The system is particularly suited for delivery of drugs having activity against the diseases located in the colon e.g. colon cancer, ulcerative colitis, protozoal infections etc.

### BACKGROUND OF INVENTION

The present invention discloses an “Improved Oral Targetted Drug Delivery System”. The system is particularly suited for delivery of drugs having activity against diseases located in the colon e.g. colon cancer, ulcerative colitis, protozoal infections etc. By way of example, data pertaining to an anti-cancer agent, 5-Fluorouracil has been provided. However, the same is purely for illustrative purposes and is not restricting.

The drug 5-Fluorouracil (5-FU) is one of the most commonly used anti-tumor agents. It was first synthesized in 1957 (Heidelberger, C., Chaudhuri, N. K., Danneberg, P., Mooren, D., Griesbach, L., 1957. Fluorinated pyrimidines, a new class of tumor-inhibitory compounds. Nature 179, 663-666.). At that time it was the only agent with clinical activity against colorectal cancer. Due to its structural resemblance to natural pyrimidines, 5-FU interferes with nucleic acid synthesis, inhibits DNA synthesis, and eventually halts cell growth (Langenbach, R. J., Dancenberg, P. V., Heidelberger, C., 1972. Thymidylate synthetase: mechanism of inhibition of 5-fluorouracil-2-deoxyuridylate. Biochem. Biophys. Res. Commun. 48, 1565-1571; Parker, W B., Cheng, Y. C., 1990. Metabolism and mechanism of action of 5-fluorouracil. Pharmacol. Ther. 48, 381-395.)

Currently it is used for different types of malignancies, such as those of the breast, head and neck.

### Limitations of 5-FU

### Kills Normal as Well as Abnormal Cells

Being a uracil analogue, it gets incorporated into RNA and DNA, leading to malfunction of these macromolecules and all rapidly growing cells, whether normal or abnormal. Therefore, the drug not only kills tumor cells, but also the rapidly growing normal cells, including gastrointestinal (GI) cells and bone marrow cells. This lack of target differentiation limits the use of the drug.

### Toxic

Toxicity is expressed as vomiting, diarrhoea, structural alteration of mucosal cells, decreased nutrient absorption, white blood cell (WBC) depression, and decreased platelet cells. The most serious toxicities are gastrointestinal toxicity, bone marrow inhibition and immunotoxicity. Gastrointestinal toxicity and bone marrow inhibition are the dose limiting factors and hamper the use of higher and possibly more effective doses of 5-FU.

### GI Damage

The gastrointestinal damage includes stomatistis, diarrhoea, denudation of villi, destruction of the integrity of mucosa, an increase of the permeability of intestine, and decrease of activities of intestinal enzymes such as thymidine-kinase. It has been reported that due to the cytotoxic effects of 5-FU on the mucous membrane, it causes mucositis when given via parenteral and intra peritoneal route (Mahood, D. J., Dose, A. M., Loprinzi, C. L., 1991. Inhibition of 5-fluorouracil-induced mucositis by oral cryotherapy. J. Clin. Oncol. 9, 449-452. and Pico, J. L., Avila-Garavito, A., Naccache P., 1998. Mucositis: its occurrence, consequences, and treatment in the oncology setting. Oncologist 3, 446-451)

### Micro Flora Disturbance

The ecological balance of normal micro flora is disturbed by the systemic administration of 5-FU. This leads to an alteration in the normal micro flora which, in turn, leads to a number of complications and multiple organ failure. This is accompanied by translocation of bacteria and diarrhoea. Moreover, there is a shift from gram (+) ve to gram (−) ve bacteria in the intestine, which increases the chances of secondary infections.

### Existing Formulations of 5-FU and Their Limitations

The existing formulations of 5-FU available in the market comprise either injections (parenteral) or capsules (oral intake). However, both the existing formulations suffer from limitations as follows:

Parenteral Route:

Wide ranging side effects due to cytotoxicity. This affects normal cells too, in addition to the cancerous cells. Since drug gets released directly into the blood stream, there is no way to avoid exposure to the normal cells and mitigate the side effects.

Oral Route:

The drug is also available for oral intake in form of capsules. However, despite availability of the formulation and theoretically predicted better patient compliance, the same has not become popular. A major limiting factor is the erratic drug release profile and also undesirable side effects such as diarrhea and GI hemorrhage leading to bloody stools. The unpredictable release, wide variation in therapeutic effects and also undesirable side-effects has led to reliance of the clinicians on the parenteral formulation, despite its limitations and side-effects.

Overcoming Limitations of the Oral Route

An innovative approach to overcoming the problem of undesirable side-effects and unpredictable release profile of the oral formulations was development of Colo-rectal targeted drug delivery systems. One such innovative system in the prior art is discussed below:

Innovative Colo-Rectal Targeted Oral Drug Delivery System as Disclosed in Prior Art

In a prior art review article (Sinha and Kumria, 2001: Polysaccharides in colon-specific drug delivery International Journal of Pharmaceutics 224 pp.19-38) one such innovative system is disclosed. It pertains to a Colo-rectal targeted oral drug delivery system. It employs ‘natural polymer coatings’ on the ‘core’ of the drug i.e. tablet in tablet. The core consists of the drug 5-fluorouracil, while the outer coat consists of natural polymers such as guar gum which can be digested only by bacteria in the colon. When the tablet is ingested, the drug 5-fluorouracil is not released at all in the stomach or small intestine, since its ‘protective coat’ of natural polymers cannot be digested. However, when it reaches the colon, specific bacteria in the colon digest the natural polymeric coat, leading to ‘targetted release’ of the drug only in the colon.

The prior art targeted delivery system proposed thus theoretically offered advantages of lesser side effects and better therapeutic profile, due to targeted release in the colon. However, when present inventors evaluated the system it was found to have a serious limitation as below:

Would the system work after the first dose of drug? The system was based on the presumption that targeted release would occur in the colon because colonic micro flora would assist in digesting ‘polymeric coating’ of the 5-FU tablet, resulting in release of the drug. It was presumed that colonic micro flora would not be affected by the drug released in the colon and only cancerous cells would be affected. However, if the micro flora got affected or there was viability loss of micro flora due to drug released in the colon, the targeted drug delivery system would fail and not work again. When next dose of the targeted drug delivery system was taken, on reaching the colon, the polymeric coating would not be digested by colonic micro flora owing to damage brought about by the first dose. This would result in failure of the colo-rectal targeted drug delivery system. This was a cause of concern to inventors as no information on this aspect was available in the prior art to best of knowledge of the inventors.

### The Problems Before the Inventors Were

1 Whether their concern regarding affect of 5-FU on ‘drug release profile’ of natural polymer based drug delivery systems (in which colonic micro flora play a critical and functional role), is genuine or not?
2. In case 5-FU did affect the colonic micro flora and thus functioning of natural polymer based, targeted drug delivery systems, how to solve the problem? How to ensure that functionality of delivery systems remained unaffected?

Thus, the challenge before the inventors was how to develop a rugged targeted drug delivery system which would work every time and not fail. The inventors tried a number of approaches and were eventually successful in developing an Improved Oral Targeted Drug Delivery System particularly suited for delivery of colon specific drugs and anti-cancer agents such as 5-Fluorouracil, which overcomes the drawbacks of the systems of the prior art.

### OBJECTS OF THE INVENTION

It is an object of the invention to disclose an Improved Oral Targetted Drug Delivery System particularly suited for delivery of drugs having activity against the diseases located in the colon e.g. colon cancer, ulcerative colitis, protozoal infections etc.

One more object of the invention is to disclose an Improved Oral Targetted Drug Delivery System comprising a unique combination of microspheres (drug+natural polymers) and probiotics in a single, pharmaceutically acceptable dosage form such as capsules.

A further object of the invention is to disclose an Improved Oral Targetted Drug Delivery System for anti-cancer drugs such as 5-FU in which the undesirable side-effects caused by the drug viz. diarrhea, vomiting, weight loss, hair loss etc. are either drastically reduced or eliminated altogether.

Yet another object is to disclose an oral drug delivery system in which the negative effects of the drug on the micro flora in the targeted region are overcome in an innovative manner by use of probiotics.

A still further object is to disclose an improved oral drug delivery system in which the limitations and drawbacks of the prior art are overcome.

### BRIEF DESCRIPTION OF THE DRAWINGS

—NIL—

### DETAILED DESCRIPTION OF THE INVENTION

### Elements of the Invention

The delivery system of the present invention comprises two elements packed in a single, pharmaceutically acceptable oral dosage form e.g. a capsule. The two elements are:
1. Microspheres: These comprise of the active drug 5-FU and natural polymers such as guar gum and xanthan gum.
2. Probiotics: These are in the form of spray dried, lyophilized powder of an appropriate probiotic e.g. Bifidobacterium species (Bifidobacterium bifidum, Bifidobacterium longum etc.)

The system was tested in animals (rats) and found to give extremely good results. It duly addressed the two fundamental questions in minds of the inventors as below:
1. Whether their concern regarding affect of 5-FU on ‘drug release profile’ of natural polymer based oral drug delivery systems (in which colonic micro flora play a critical and functional role), is genuine or not?
2. In case colonic drugs such as 5-FU do affect the colonic micro flora and thus functioning of natural polymer based, oral targeted drug delivery systems, how to solve the problem? How to ensure that functionality of delivery systems remained unaffected?

Significance: Since colonic bacteria play a critical and functional role in natural polymer based drug delivery systems, there was a need to minimize and if possible eliminate the negative effects of 5-FU on micro flora. Otherwise, once the drug killed the micro flora (bacteria), the system would not work after the first time, as the outer coat of natural gums would not be digested.

Prior art status: It is known in prior art that systemic administration of 5-FU disturbs ecological balance of normal intestinal micro flora, which in turn can lead to diarrhoea and other complications (Gutheli, J. C., Kearns, C. M. (Eds), 2008. Gastrointestinal complication of chemotherapy in M. C. The Chemotherapy Source Book, Lippinicott Williams & Wilkins, USA, pp. 197-208). Suppression of normal micro flora by 5-FU may lead to reduced colonization resistance with subsequent overgrowth of pre-existing, naturally resistant microorganisms like Clostridium difficile in GIT.

However, what has not been disclosed in the prior art is “Whether 5-FU will affect specific microbes/micro flora involved in digestion of natural gums, thus affecting release of ‘natural gum based’ drug delivery systems?” Inventors found that surprisingly this aspect had not been addressed at all in the prior art and no experimental evidence or data was available in this regard.

Challenges: It is relevant to mention here that it is not practically feasible to isolate from a highly complex colonic microbial population, only a specific class of polysaccharide metabolising bacteria (responsible for the release of drug from the coated tablets) and evaluate it regarding its sensitivity towards 5-FU. A suitable biological experimental system had to be innovatively designed which provided clear-cut technical evidence as to whether an anti-cancer drug such as 5-FU affects release profile of natural gum based drug delivery systems.

### Experimental Approaches

Experimental approaches involved both in vitro and in vivo studies. These were focused on two aspects:
a. To evaluate whether an anti-cancer drug such as 5-FU affects drug release profile of natural gum based drug delivery systems.
b. To demonstrate a practical strategy to overcome the negative affects of the drug on micro flora, if any

To answer the questions as above, the inventors adopted an innovative experimental approach using a ‘living biological system’ comprising rats. All the experimental procedures were approved by the Institutional Animal Ethics Committee vide Approval Number 954/ac/06/CPCSEA/09/19. The experimental study was conducted on thirty Wister Albino rats. The animals were divided into 6 groups of five each. Each animal in the group was given oral dose as indicated in Table 1 below:

TABLE 1
Treatments and dosages given to animals (Wister Albino Rats)

| S. No. | Groups | Treatment | 5-FU Dose (p.o.) |
|---|---|---|---|
| 1. | Group I | Microspheres of xanthan and | 8 mg/day |
| | | guar gum alone | |
| 2. | Group II | 5-FU powder | 8 mg/day |
| 3. | Group III | 5-FU microspheres (50% | 8 mg/day |
| | | loading capacity at 16 mg) | |
| 4. | Group IV | Probiotics | 1 g/day |
| 5. | Group V | 5-FU powder along with | 8 mg/day + 1 g/day |
| | | probiotics | |
| 6. | Group VI | 5-FU microspheres along with | 8 mg/day + 1 g/day |
| | | probiotics | |

The dosages were freshly prepared every day by suspending in milk. They were administered by oral gavage needle for first and subsequent exposure for a duration of five days each, at an interval of 20 days.

### Experimental Approach A

2 animals from each group were humanely sacrificed for the collection of caecal contents in sterile petri plates, which was immediately utilized for the dissolution studies. For dissolution studies, compression coated tablets of 5-FU with natural gums and microspheres of 5-FU with natural gums were used. The experiments were carried out in 250 ml beaker immersed in water maintained in the jars of dissolution test apparatus. Initial studies were carried out in 150 ml of 0.1N HCl (pH 1.2) for 2 h. After this, NaOH was added to the dissolution media and the pH was adjusted to 6.8. The volume was made up to 200 ml with phosphate buffer of pH 6.8. The study was continued for 3 hours after which, caecal contents (4% w/v) were added. Dissolution in the caecal content media was carried out till completion of 24 hours. The results of the studies are given in Table 2 below:

TABLE 2
Effect of caecal contents of rats given 5-FU orally, on Drug Release
Profile of oral, targeted drug delivery systems

| | | Oral, targeted drug delivery system evaluated under | |
|---|---|---|---|
| | | in vitro conditions | |
| | | (% drug released in 24 h on exposure to caecal | |
| | Treatment given to rats | contents of the rat) | |
| | whose caecal contents were | Compression coated | 5-FU loaded microspheres |
| S. | used for evaluating drug | tablets of 5-FU coated | comprising the drug and |
| No. | release profile | with natural gums | natural gums |
| 1. | Control- No drug, only | 83 | 92.20 |
| | excipients (Micro spheres of | | |
| | natural gums viz. XG:GG) | | |
| 2. | 5-FU powder alone | 52 | 41.56 |
| 3. | 5-FU microspheres alone | 54 | 51.36 |

### The Following Answer was Obtained from the Experiments Conducted

5-FU considerably damages micro flora involved in release of drug from the ‘natural gum based’ delivery systems: Caecal contents of rats fed on 5-FU powder or 5-FU microspheres did not contain sufficiently active microbes. This is proven by the fact that when natural gum based drug delivery systems (compression coated tablets/microspheres) were exposed to the caecal contents of such rats, drug release was drastically reduced, ranging from 41%-54% compared to 83-92% in the control in which rats were not given any drug orally. This indicated that ‘micro flora’ in rats fed orally with 5-FU had been ‘damaged’ because it is these micro flora (bacteria) which are involved in digesting the natural gums in the ‘targetted drug delivery systems’. Since release of drug was very less as compared to control, it indicated that the bacteria were being killed by the drug.

Thus the first question in the minds of the inventors i.e. “Whether their concern regarding affect of 5-FU on ‘drug release profile’ of natural polymer based oral drug delivery systems (in which colonic micro flora play a critical and functional role), is genuine or not?” stood answered, positively.

Yes, micro flora were being affected and this affected ‘drug release profile’ of natural gum based drug delivery systems, since adequate number of micro flora were not there to digest the ‘gums’.

This indicates that drugs with antimicrobial activity (both in conventional as well as colon targeted form) adversely affect colonic bacteria. Thus, ‘targetted drug delivery systems’ based on natural gums were unlikely to succeed technologically as their functionality was based on the intactness of micro flora, which was practically very difficult if patient was taking antibiotics or any other drug which affected micro flora.

Now that the inventors had been able to prove that ‘colonic micro flora’ was being affected by drugs and ‘drug release’ of colon targeted oral drug delivery systems was also considerably reduced, the second question viz. “In case colonic drugs such as 5-FU did affect the colonic micro flora and thus functioning of natural polymer based, oral targeted drug delivery systems, how to solve the problem? How to ensure that functionality of delivery systems remained unaffected?” had to be answered.

After sustained research investigations and various experimental approaches, the inventors solved the problem in a very innovative and practical manner using two technical interventions:
i) Administration of 5-FU to test animals, in form of microspheres comprising ‘drug+natural gums’ instead of powder alone:
Instead of giving 5-FU in active form to the test animals, it was given in form of microspheres comprising mixture of 5-FU and natural polymeric gums viz. guar gum and xanthan gum (details of microsphere preparation duly given in Table 4). Thus, as compared to prior art in which coating of 5-FU tablets with natural polymers has been disclosed, the inventors used natural polymer based microspheres of 5-FU which has not been disclosed in the prior art. Use of microspheres was attempted because of two reasons—Firstly to enhance ‘drug dispersal’ in the colon so that undesirable side-effects at the site of release could be reduced. Experimental evidence proved that use of microspheres significantly reduced the side effects as compared to 5-FU powder. Secondly, to simplify the manufacturing process and avoid the need for any specialized machinery. Unlike the ‘tablet-in-tablet’ delivery system of the prior art which requires specialized machinery, the ‘microsphere based delivery system’ of present invention does not require any specialized machinery and industrial scale batches can easily be manufactured.
ii) Co-administration of commercially available probiotics along with 5-FU microspheres:
To ‘buffer’ colonic micro flora from 5-FU damage, the inventors evaluated whether co-administration of probiotics alongwith the drug delivery system would have a ‘replenishing’ effect on colonic micro flora which had been damaged/lost due to ‘chemical attack’ of the drug. Commercially available powdered form of Bifidobacterium species (Bifidobacterium bifidum and Bifidobacterium longum) was co-administered alongwith microspheres of 5-FU to test animals (Albino Wister Rats). A dose of 1 g of probiotics was co-administered per animal/per day alongwith 5-FU microspheres dose of 8 mg/animal/day. This was done to confirm whether the negative side-effects on the gut micro flora by microspheres of 5-FU could be overcome by co-administration of probiotics. Results are given in Table 3 below:

TABLE 3
Effect of caecal contents of rats given 5-FU and probiotics orally,
on Drug Release Profile of oral, targeted drug delivery systems

| | | Oral, targeted drug delivery system | | |
|---|---|---|---|---|
| | | evaluated under in vitro conditions | | |
| | | (% drug released in 24 h on exposure | | Remarks |
| | | to caecal contents of the rat) | | Microspheres |
| | Treatment given to rats | | 5-FU loaded | offer ease of |
| | whose caecal contents | | microspheres | manufacture |
| | were used for | Compression coated | comprising | and packing |
| S. | evaluating drug release | tablets of 5-FU coated | the drug and | into a single |
| No. | profile | with natural gums | natural gums | dosage form |
| 1. | Control 1- No drug. | 83 | 92.20 | Drug release |
| | Only excipients (Micro | | | profile of |
| | spheres of natural gums | | | microspheres |
| | viz. XG:GG) | | | is superior |
| | | | | compared to |
| | | | | tablets (92% |
| | | | | vs 83%) |
| 2. | Control 2- No drug. | 92 | 94.32 | Drug release |
| | Only probiotics | | | profile of |
| | | | | microspheres |
| | | | | is superior |
| | | | | compared to |
| | | | | tablets (92% |
| | | | | vs 83%) |
| 3. | 5-FU powder alone | 52 | 41.56 | Drug release |
| | | | | is drastically |
| | | | | reduced on |
| | | | | treatment with |
| | | | | 5-FU |
| 4. | 5-FU microspheres alone | 54 | 51.36 | Drug release |
| | | | | is drastically |
| | | | | reduced on |
| | | | | treatment with |
| | | | | 5-FU |
| 5. | Probiotics + 5-FU powder | 70 | 85.36 | Probiotic co- |
| | | | | administration |
| | | | | helps |
| | | | | overcome the |
| | | | | adverse effect |
| | | | | of drug on |
| | | | | micro flora |
| 6. | Probiotics + 5-FU | 76 | 92.32 | Probiotic co- |
| | microspheres | | | administration |
| | | | | helps |
| | | | | overcome the |
| | | | | adverse effect |
| | | | | of drug on |
| | | | | micro flora |

### Hypothesis

The approach adopted by the inventors was based on the hypothesis that if ‘micro flora’ of colon was regarded as a single ‘unit’ and this unit could somehow be protected from 5-FU damage, then the specific class of polysaccharide metabolizing bacteria responsible for the release of drug from the coated tablets would also be automatically protected. This would lead to success of any ‘natural polymer based’ colon targeted drug delivery system in which colonic microbes play a critical and functional role.

### Experimental Approach Adopted and Results Obtained

When this was tested experimentally, results obtained indicated that this thinking was correct (Table 3). In microspheres exposed to caecal contents of rats fed on 5-FU alone, the drug release was only 41.56% whereas in microspheres exposed to caecal contents of rats fed on 5-FU+probiotics, the drug release increased dramatically and was 92.32% !

### Interpretation

Co-administration of probiotics can help to overcome the adverse effects of the drug on micro flora almost completely. This is proven by the fact that in vitro drug release from microspheres exposed to caecal contents of rats fed 5-FU+probiotics (92.32%) was very close to that of the controls (92-94%) in which animals had not been given any drug. The results of the above studies thus provided first direct experimental evidence regarding two aspects:
1. Colonic Drugs such as 5-FU affected the gut micro flora adversely: Hence, failure of targeted drug delivery systems using natural polymers could not be ruled out because the very basis of functionality of such ‘natural polymer based targeted drug delivery systems’ was intactness of colonic micro flora.
2. Damage to micro flora can be overcome: The damage to colonic micro flora can be overcome by co-administration of appropriate probiotics.

To address whether the unwanted side-effects of colonic drugs e.g. diarrhea, hair loss etc. could also be reduced or eliminated using the ‘innovative approach’ of the present invention, trials were conducted in animals (rats) using 5-FU as a ‘model’. Results of the studies are given below:

Unwanted Side-Effects on Oral Administration of 5-FU Powder

Albino Wister Rats given 5-FU powder (8 mg/day) for 5 days suffered from loss of hair, body weight and also bloody diarrhea as revealed by examination of the caecal contents obtained after sacrificing the animals. Histopathological examination of the colon showed ulceration and loss of epithelial lining. Occurrence of diarrhea and mucocitis in the animals indicated that micro flora of the colon was completely disturbed.

Unwanted Side-Effects on Oral Administration of 5-FU Microspheres

Parallel studies using ‘natural polymer’ based microspheres of 5-FU (targeted drug delivery systems) revealed that though side-effects in comparison to 5-FU powder were less, they were not eliminated, indicating disturbance of micro flora was occurring. The rats given polymer coated 5-FU microspheres did not suffer from hair loss though weight loss and diarrhea did take place. However, traces of blood were not present in the caecal contents obtained by sacrificing the animals. Histo-pathological examination of the colon revealed focal ulcerations only. This was in contrast to the group which had been given 5-FU powder in which diffuse ulceration of the colonic mucosa with loss of lining epithelium was observed.

Unwanted Side-Effects on Oral Administration of 5-FU Microspheres+Probiotics

Concomitant administration of probiotics alongwith 5-FU microspheres completely eliminated the negative side-effects of the drug and significantly protected ‘colonic micro flora’ in the test animals. This was proven by the following observations:
i) There was no occurrence of diarrhea at all and colonic/caecal contents of the test animals were normal
ii) The test animals did not suffer from any weight loss and their weight was same as compared to the control animals
iii) Histo-pathological examination of the colon of test animals revealed that mucosal and serosal layer of colon was normal without any loss of epithelial lining.

The present invention thus marks a turning point related to:

### a. Successful Performance of Targeted Drug Delivery Systems

More specifically it has proven success of ‘oral colon targeted drug delivery systems using natural polysaccharide approach, by overcoming ‘defects’ in systems of the prior art. It discloses for the first time that prior art ‘colon targetted drug delivery systems based on natural polymer approach’ are technically flawed. Drug release of such systems will be severely compromised after ingestion of the first dose since the drug will affect the microflora, which are involved in ‘functionality of the drug delivery system based on natural polymers’. It is these micro flora which digest the outer natural polymer coat of the targeted drug delivery systems, leading to release of the drug. If they themselves are killed by the drug released, coat will not be digested when next dose is taken and system will fail. This limitation has been experimentally demonstrated by the inventors and overcome in an innovative manner by co-administration of probiotics. Compared to systems of prior art in which drug release was only 41.56%, the drug release in system of present invention was 92.32%! Table 3.

### b. Drastic Reduction in Undesirable Side-Effects of Drugs Targeting the Colon

The undesirable effects of colon targeted drugs e.g. anti-cancer drugs such as 5-FU can surprisingly be overcome by use in ‘microsphere’ form alongwith natural gums and also co-administration of probiotics. This has been experimentally demonstrated by the inventors in an animal model (rat). Animals co-administered probiotics alongwith the anti-cancer drug 5-FU did not suffer from bloody diarrhea and loss of weight/hair while animals in which probiotics were not given suffered from undesirable effects of bloody diarrhea and also loss of weight and hair.

TABLE 4
Method of Preparation of natural polymer coated 5-FU
microspheres - emulsion polymerization technique

| S. | Description of | |
|---|---|---|
| No. | process stage | Remarks |
| 1. | Mixing of | Natural polymeric mixture viz. Guar gum and |
| | constituents | xanthan gum mixture (1 g each) is dispersed |
| | | in 200 ml of water containing 100 mg of the 5-FU |
| | | and tween 80 (0.2% w/v). |
| 2. | Swelling and | Mixture is allowed to swell for two hours. |
| | acidification | Concentrated sulphuric acid (0.2% w/v) is |
| | | added and aqueous phase is poured into castor oil. |
| | | Antifoam A in a concentration of 0.1% w/v is |
| | | added to it. Glutaraldehyde (2 ml) is added under |
| | | stirring conditions. |
| 3. | Agitation | The system is kept under agitation for 4 hours |
| | | using stirrer at a speed of 4000 rpm. |
| 4. | Microsphere | The microspheres are collected by centrifugation |
| | collection, | at the speed of 2500 rpm, after washing thrice |
| | washing and | with isopropyl alcohol and dried in a vacuum |
| | drying | desiccator for 48 h. |

### NOVELTY ASPECT OF THE INVENTION

The Improved Oral Targeted Drug Delivery System (O-TDDS) for delivery of colon specific drugs and anti-cancer agents such as 5-Fluorouracil, comprising a combination of microspheres (mixture of drug+natural polymers such as guar gum and xanthan gum) and probiotics in a single, pharmaceutically acceptable oral dosage form such as a capsule, has not been disclosed anywhere in the prior art to best of knowledge of the inventors. It is thus novel.

### INVENTIVE STEP

The inventive step thus lies in the successful development by the inventors of an Improved Oral Targeted Drug Delivery System for delivery of colon specific drugs and anti-cancer agents such as 5-Fluorouracil, comprising a combination of microspheres (mixture of drug+natural polymers such as guar gum and xanthan gum) and probiotics in a single, pharmaceutically acceptable oral dosage form such as a capsule. It represents a significant technical advance over existing knowledge.

Further, the inventive step lies in the complete elimination of the negative side-effects of the drug (e.g. 5-FU) on the micro flora of the colon. In the prior art, since the problem of affect of 5-FU on micro flora had not been addressed, such targeted drug delivery systems in which micro flora played a critical and functional role, were technically flawed with a strong possibility of failure. This was because once the micro flora was upset; the next dosage form would not work as the natural polymer coat would not open due to lack of colonic bacteria to digest the same. This problem has been solved in the present invention.

### INDUSTRIAL APPLICATION

The present invention discloses an improved oral targeted drug delivery system particularly suited for delivery of drugs having activity against the diseases located in the colon e.g. colon cancer, ulcerative colitis, protozoal infections etc.

The same can be manufactured on an industrial scale easily because readily available, off-the-shelf constituents have been utilized in the system. Manufacturing and scale-up is thus simple, facilitating industrial application.

### Advantages of the Present Invention

1. Drastically reduced side-effects

2. Better patient compliance

3. Better therapeutic profile

4. Compact and effective dosage form

5. Economy of manufacturing aspects

The concept disclosed in the present invention can well be extrapolated to other drugs. It can be used to reduce the side-effects in delivery systems where natural gums/polymers have been utilized to bring about ‘targeted delivery’. Examples of such drugs targeted to colon using combination of natural gums such as guar gum and xanthan gum or guar gum alone are given in Table 5 and Table 6 respectively, below:

TABLE 5
Drugs targeted to colon using combination of guar gum
and xanthan gum

| S. | | |
|---|---|---|
| No. | Drug used | Reference/s |
| 1. | 5- | Sinha, V. R., Kumria, R., 2001. Review: |
| | Fluorouracil | Polysaccharides in colon-specific drug delivery. Int. |
| | | J. Pharm. 224, 19-38. |
| | | Sinha, V. R., Singh, A., Singh, S., Bhinge, J. R., |
| | | 2007. Compression coated systems for colonic |
| | | delivery of 5-fluorouracil. J. Pharm. Pharmacol. |
| | | 59, 359-365. |
| 2. | Cimetidine | Wang, D., Larrabeo, S., Clifford, K., Trmblay, J., |
| | | Friend, D. R., 1997. USP dissolution apparatus |
| | | (reciprocating cylinder) for, screening of guar gum |
| | | based colonic delivery formulation. J. Cont. Rel. |
| | | 47, 173-179. |
| 3. | Aceclofenac | Ganesh, K., Prashant, G., Das, S., 2010. |
| | | Formulation and in-vitro evaluation of colon |
| | | targeted drug delivery of Aceclofenac. J. Pharm. |
| | | Res. 3, 1082-1086. |

TABLE 6
Guar Gum based Colon-specific formulations

| S. No. | Drugs used |
|---|---|
| 1. | Budenoside/Dexamethasone |
| 2. | Dexamethasone |
| 3. | Albendazole |
| 4. | Indomethacin |
| 5. | Technetium-99m-DTPA |
| 6. | 5-ASA |
| 7. | Mebendazole |
| 8. | Albendazole/Tinidazole |
| 9. | Celecoxib |
| 10. | 5-Fluorouracil |
| 11. | Trimetazidine dihydrochloride |
| 12. | Ornidazole |
| 13. | Metoprolol tartrate |
| 14. | Theophylline |
| 15. | Mesalazine |
| 16. | Sennoside |
| 17. | Rofecoxib |
| 18. | Metotrexate |
| 19. | Diltiazem hydrochloride |

Thus, present invention has successfully demonstrated a practical mechanism to maintain the integrity and intactness of the intestinal/colonic micro flora, in face of ‘chemical attack’ by colon specific drugs listed in Table 6 above. Since the approach used in the present invention primarily involves use of microspheres of the drug+probiotics ‘packed’ in a single, oral, pharmaceutically acceptable dosage form, the example of 5-FU given is not restrictive but may be regarded as an illustrative example to better explain the invention and promote understanding of the same.

Once the innovative concept as disclosed in the present invention is understood, embodiments, deviations and improvements to the same can easily be carried out by those skilled in the art e.g.
Several drugs+single probiotic: Instead of single drug, multiple drug combinations can be used along with single probiotic.
Different drugs: Delivery systems can utilize any suitable drug apart from drugs disclosed in present application.
Several drugs+different probiotic: Any other probiotic apart from probiotic disclosed in present application can be used.
Single drug+multiple probiotics: More than one probiotic can be used in a system.
Multiple drugs+multiple probiotics: A combination of more than one drug and more than one probiotic can be used.

Accordingly, any such embodiments, deviations or improvements should be regarded as within the scope of the present invention.

I claim:

1. An improved Oral Targetted Drug Delivery System (O-TDDS) wherein the same comprises two elements viz.
i. microspheres which are mixtures of the drug and natural polymers
ii. probiotics in powdered form
packed together in a single, pharmaceutically acceptable oral dosage form such as a capsule.

2. The drug delivery system of claim 1 wherein the drug is any drug selected from the group comprising known anti-cancer compounds such as 5-Fluorouracil or any other anti-cancer compound.

3. The drug delivery system of claim 1 wherein the drug is any drug selected from the group comprising colon specific compounds such as Budenoside/Dexamethasone, Dexamethasone, Albendazole, Indomethacin, 5-ASA, Mebendazole, Albendazole/Tinidazole, Celecoxib. Trimetazidine dihydrochloride, Ornidazole, Metoprolol tartrate, Theophylline, Mesalazine, Sennoside, Rofecoxib and Methotrexate.

4. The drug delivery system of claim 1 wherein the drug is the anti-cancer agent, 5-Fluorouracil.

5. The drug delivery system of claim 1 wherein the natural polymers are gums such as guar gum or xanthan gum or any other natural polymer.

6. The drug delivery system of claim 1 wherein the probiotics are any suitable probiotic including but not restricted to Bifidobacterium species such as Bifidobacterium bifidum and Bifidobacterium longum.

7. An improved oral targeted drug delivery system as substantially described herein with reference to the detailed description.
